(19) United States
(12) Patent Application Publication
(10) Pub. No.: US 2010/0070051 A1
(43) Pub. Date: Mar. 18, 2010

(54) PROSTHESIS SHAFT WITH ACTIVE AIR RELEASE

(75) Inventor: Felix Carstens, Weinstrasse (DE)

Correspondence Address: WHITHAM, CURTIS & CHRISTOFFERSON & COOK, P.C., 11491 SUNSET HILLS ROAD, SUITE 340, RESTON, VA, 20190, US

(73) Assignee: OTTO BOCK HELATHCARE GMBH, DUDERSTADT (DE)

(21) Appl. No.: 12/515,603

(22) PCT Filed: Nov. 11, 2007
(86) PCT No.: PCT/DE07/02064

§ 371 Date: May 20, 2009

(51) Int. Cl. A61F 2/80

(52) U.S. Cl. 623/34; 623/36

(57) ABSTRACT

The invention relates to a cup-shaped prosthesis shaft consisting of an air-tight and essentially rigid material comprising a sealing means for protecting against the entry of air from the proximal open side, said shaft being used in prostheses for replacing limbs. The inside of the shaft is provided with at least one pump device such that relative movements between the stump and the shaft during the use of the prosthesis pump air out of the inside of the prosthesis shaft via flow channels and monoway valves to the outside of the prosthesis shaft. The pump action is obtained by varying the volume of a compartment formed from flexible or elastic films, said compartment being arranged adjacently to the strump in the main extension thereof. The compartment is either fixed to the inner wall of a rigid prosthesis shaft, or built into the wall of a flexible and elastic removable stump coating which is arranged, as part of the prosthesis shaft, between the stump and a rigid frame.

The invention relates to a prosthesis socket for use in an artificial replacement for an amputated limb, according to the preamble of claim 1.

### GENERAL INTRODUCTION

In prosthesis manufacture, a cup-shaped container made from substantially flexurally stiff material is generally molded to match the stump and, in accordance with the rules of prosthetics, is shaped in such a way as to permit painless transfer of forces via the amputation stump through this so-called prosthesis socket. In the case of a prosthesis to compensate for loss of part of the upper limb, the forces that are to be transferred via the socket are generally pressure forces and tensile forces and moments that arise on the hand during use of an arm, for example. If the prosthesis compensates for loss of part of the lower limb, the forces arise primarily from the static and dynamic transfer of the body weight during locomotion. In addition to these pressure forces and bending and rotation moments, however, leg prostheses are also subject to tensile forces that tend to remove the prosthesis from the stump. This results already from the inherent weight of the prosthesis since, when the prosthesis is lifted from the ground, this weight exerts a pull on the leg stump. There are in addition the dynamic forces that arise to an appreciable extent during walking and running.

Various techniques are known for securing a prosthesis safely on a stump and for counteracting the withdrawal forces, and the most common of these methods will be briefly outlined here for general understanding: securing by means of belts and straps, anchoring on undercuts of bone protuberances, use of a stocking-like elastic cover which is made of an airtight elastomer and which is rolled over the stump so as to bear fully on the skin, as a result of which this cover sits securely on the stump both by means of underpressure and also by means of friction, and which is connected to the prosthesis socket in a mechanically releasable manner, and, finally, the use of underpressure in the socket between the skin of the stump and the inner socket wall, so as to create what are called suction sockets. Nowadays, in modern prosthesis manufacture, it is state of the art to adapt the prosthesis sockets wherever possible exactly to the volume and anatomical shape of the stump, taking into account the known aspects of specifically modeled socket shapes, in such a way that the pressure of the prosthesis socket is distributed over the largest possible surface area of the stump upon loading, and in such a way that as far as possible no air gap is present between the stump and the inner face of the socket. However, it is also known that the volume of an amputation stump can change considerably as a result of displacement of body fluid. In particular, the abovementioned full-contact sockets, which are so advantageous in other respects, have the effect that, as a result of the desired uniform pressure on the stump during the loading phase, which pressure is naturally greater than the normal ambient pressure, there is considerable displacement of fluid in the stump and, consequently, the volume of the stump decreases. A stump whose volume is reduced in this way not only permits undesired movements between stump and socket, so-called stump/socket pseudarthroses, but can very quickly lead to considerable problems for the prosthesis wearer, since the desired uniform pressure distribution is no longer ensured and since the movement between stump and socket can lead to rubbing and pressure sores. In the case where the prosthesis is bound to the stump by underpressure and, without additional measures, the skin of the stump is intended to form, together with the inner face of the socket wall, a seal against entry of air, as is very often the case in above-knee prostheses, the loss of stump volume very often also means the loss of the secure airtight connection to the socket. If air is able to penetrate into the socket, the worst-case scenario entails sudden uncontrolled slipping of the prosthesis from the stump, and, at the very least, a noticeable noise may develop due to the admitted air being pressed back out again in the proximal direction between the skin and the socket wall upon loading of the prosthesis.

The present invention relates specifically to binding of the prosthesis by the suction effect, the principle according to the invention being independent of the type of proximal prosthesis seal, and the invention thus being applicable in principle to all types of suction prostheses at different amputation heights.

### PRIOR ART

The binding of prostheses to an amputation stump by means of underpressure is today a commonly used method, and the various techniques involved are known to specialists in this field. What is called a prosthesis socket is generally adapted individually to the stump conditions, according to the generally recognized rules of prosthetics, in such a way that the stump completely fills the cup-shaped socket, which is closed at the bottom end. The material used for such sockets nowadays is generally a thermoplastic or a fiber composite. Accordingly, the sockets are thermally formed on a positive model of the prosthesis socket or are cast over such a model with liquid plastic and fiber layups between films. This results in a substantially dimensionally stable cup-shaped socket which is airtight or can be made airtight. When the stump is inserted into such a socket, it naturally displaces the air located in the socket. If suitable measures are taken to ensure that no air can flow into the socket, the socket is sucked like a plunger firmly onto the stump and cannot therefore be readily withdrawn again, for example not without opening a valve arranged distally in the socket. The sealing against unwanted entry of air can be afforded by various measures. In stumps with a large proportion of soft tissue, for example in many above-knee stumps, the uninterrupted contact of the skin with the inner wall surface of the prosthesis socket is often sufficient. For suction prostheses in the below-knee area, where the bone structures lie directly beneath the skin and where there is usually insufficient sealing between the skin of the stump and the inner wall of the socket, hose-like and airtight elastic covers are usually pulled up from the outer face of the prosthesis socket and over the knee as far as the thigh. Another possibility is the formations of sealing lips, which are described, for example, in documents DE 10142491 B4 or DE 10142492 A1.

In the present state of the art, the objective of most suction sockets is simply to secure the prosthesis safely on the amputation stump by means of an underpressure, which arises purely passively through tensile forces acting on the prosthesis and which is not present when the prosthesis is loaded. Only Carl Caspers et al. set out to actively lower the pressure in a prosthesis socket to below atmospheric pressure, that is to say to create an active underpressure. Mention should be made here in particular of documents U.S. Pat. No. 5,549,709, U.S. Pat. No. 5,904,722 and US 2002/0091449 A1, for example, which, like those also referred to above, are intended to be incorporated by reference into this application. Caspers found that a socket pressure lowered to below atmospheric pressure is not only advantageous for binding the prosthesis to the stump, but also has the effect of keeping the stump volume constant, since the underpressure, even during the loading phase of the prosthesis, counteracts the displacement of body fluid caused by the load pressure exerted on the skin, and it does so because the suction action prevents the skin of the stump from detaching from the firm inner surface of the prosthesis socket. Therefore, the stump is as it were sucked into the individually adapted volume of the prosthesis socket. In addition to the aforementioned reduction in the fluctuations in the volume of the stump, the suction action of the underpressure results in reduced relative movement between socket and skin (reduced stump/socket pseudarthrosis), improved rotation stability of the prosthesis, more exact guiding of the prosthesis and, not least, also improved proprioception for the prosthesis wearer, with a correspondingly reduced foreign body sensation and enhanced security.

Caspers describes the use of an active underpressure pump, which is connected in terms of flow to the prosthesis socket, for generating a vacuum between the skin of the prosthesis wearer and the interior of the socket. Mechanical pumps are built into the prosthesis tube, which forms the connection between prosthesis socket and artificial foot, or a mechanical pump is integrated into a resilient part of an artificial foot. The pump is in any case provided outside the interior of the socket and is connected to said interior of the prosthesis socket via a flow channel, in the simplest case via a hose. The pumps are activated by virtue of the fact that the cyclic loading and unloading of the prosthesis during walking is able to provide active pumping work. For this purpose, a relative movement is needed between at least two parts of such a mechanical pump. In the aforementioned installation variants, this relative movement is afforded by the fact that the pump functions as an impact damper and shortens slightly under axial loading and, when the load is removed, returns to its starting position, for example under a spring force. A system is also known in which a mechanical pump of this kind is secured on the rear (the dorsal face) of the prosthesis tube, as seen in the direction of walking, and in which a cylinder is connected fixedly to the prosthesis tube, while a piston rod of a piston located in the cylinder protrudes downward and is actuated by the up and down movement of the heel part of a movable artificial foot during the cyclic loading and unloading of the foot while walking. Recently, an electrically driven pump has also been used to generate an active underpressure in the prosthesis socket and is available on the American market under the trade name TSS Vaculink.

All of the previously known systems have in common that an external mechanical pump is connected to the interior of the socket via a flow channel and, if appropriate, a corresponding valve system.

The systems according to the prior art have the disadvantage of, on the one hand, being susceptible to mechanical failure and, on the other hand, of entailing a not inconsiderable additional weight, which has to be carried around by the prosthesis wearer and has to be accelerated and decelerated on each step, which requires considerable additional energy. In the case of the electric pump, the required energy has to be made available in the form of batteries or accumulators such that, over and above the additional weight, there is also the factor of having to carry around replacement batteries and of changing the batteries when necessary, or of always having to hand a suitable mains charger in order to be able to replace the electrical energy that has been used up. All of this is very cumbersome and also limits the mobility of the prosthesis wearer. Moreover, the necessary maintenance work and, if appropriate, the replacement of consumables such as batteries, entails additional costs.

Because of their high mechanical demands and their complexity, all of the known systems are so expensive that they considerably increase the overall costs of a prosthesis and therefore, for cost reasons alone, are used only for a small number of prosthesis wearers. A further disadvantage of the systems according to the prior art is that, in certain configurations, only certain components can be used for the prosthesis set-up, for example only a quite specific design of the artificial foot, which greatly restricts the design options open to the prosthesis manufacturer.

To permit the increase in volume, all pumps arranged on the outer face of a prosthesis socket have to overcome the forces that act on the mutually movable parts of the pump as a result of the built-up pressure difference. For example, if a ball pump made from a flexible material with restoring force is used to evacuate a space, the attainable underpressure depends substantially on with what force the pump restores itself after a reduction in volume. In this type of use for suction, a typical ball pump, starting from a defined pressure difference, will no longer be able to return to its starting state and instead will remain collapsed on account of the suction forces. By contrast, the arrangement of the pump according to the invention in the interior of the prosthesis socket is of great advantage because the volume-increasing force only has to compensate for flow/pressure losses and does not have to work against the pressure difference between the interior of the socket and the atmosphere, since of course the higher atmospheric pressure does not act on the outer face of the flexible wall of the pump chamber. In this way, only very little force is needed for restoring the pump chamber in the direction of an increase in volume, after the preceding reduction in volume by pressure forces. This force can be applied, for example, by a simple foam insert, a small spring, a flexible chamber material with restoring force, or a Velcro connection for transmitting tensile forces. In this way, the pump can be made very thin and from very lightweight materials without high mechanical demands, which is of great advantage for the use in prostheses. Since in the opposite direction, that is to say in the direction of a reduction in volume of the pump space, the pressure difference against which the pump has to work and also the restoring spring forces, the necessary forces for pumping in the arrangement according to the invention are correspondingly lower, which is a further advantage and serves to avoid pressure problems on the stump.

### OBJECT OF THE INVENTION

Following what has been said above, the object of the invention is to create a simple system for reducing the pressure in the interior of a socket between the skin of the stump and the inner wall of the socket, which system is not susceptible to mechanical failure, does not add any appreciable additional weight to the prosthesis weight, requires only minimal maintenance if any, does not need any external energy, can be used universally and is less expensive than the systems according to the prior art, such that a far greater number of amputees can benefit from the advantages of actively reduced pressure in the socket.

The object of the invention is achieved by the features set forth in claim 1, with different embodiments being represented by one of the dependent claims or by a combination of several dependent claims. The measures according to the invention result in a system with which the pressure in the gap between an amputation stump and a prosthesis socket receiving the stump is lowered to below the level of the atmospheric pressure, without this requiring complex and heavy additional components outside of the prosthesis socket. According to the invention, the energy needed for displacing gas (air) counter to a higher pressure is derived from the kinetic energy during use of the prosthesis. The components according to the invention are for the most part made of airtight film and open-cell foam or are foamed by two layers (63, 64) of an elastic stump cover (55), which form a chamber (34) in the wall of the stump cover. They are inexpensive and their weight is almost negligible, both of which aspects are of enormous advantage over the prior art. Since the main components are located in the socket interior, the prosthesis according to the invention is scarcely distinguishable in appearance from conventional prostheses of known design, and no bulky additional parts have to be integrated into the cosmetic picture. Apart from the nonreturn valve (16) with the connection element (14) and the flow channel (15) and possibly a second release valve (52), no additional components are needed outside the socket (2) in order to implement the invention. A prosthesis having the properties of the present invention can accordingly be set up with all known combinations of adapter pieces and other components, and the prosthesis manufacturer does not have to use components specifically adapted to the vacuum system, for example particular artificial feet. All of these stated features represent considerable advantages over the prior art.

### BRIEF DESCRIPTION OF THE INVENTION

In a particularly preferred embodiment of the invention, at least one flexible chamber (13), which is formed from films (31, 32) and is preferably filled with an open-cell foam with high restoring force (35), is secured at a strategically favorable position in the interior (8) of the socket in such a way that the stump movements in a gap (26) present between stump and socket cyclically compress and expand the restorable foam in the chamber, and thus the chamber itself, through the natural movements resulting from the use of the prosthesis. A one-way valve (37), which is preferably formed by film (48) as a flutter valve, allows air from the gap (26) between stump and socket to be sucked into the chamber (13) or pump space (34) on account of the restoring force of the foam (35) acting on the chamber (13) during the unloading phase and the resulting volume increase of the pump space (34) formed by the chamber.

Instead of a chamber made from thin, flexible film without dimensional stability, the chamber can also be made from a flexible material with restoring force, as a result of which the foam insert can be omitted. Instead of foam, a second air chamber of smaller volume can also be used to generate the required restoring forces. If the filling volume and therefore the pressure in this second chamber is designed such that it can change, the restoring force can be changed by the change of the filling pressure of the second chamber.

The chamber (13) is connected in flow terms to the atmosphere via a second one-way valve (16). In the loading phase of the prosthesis, when a force is again applied from the stump onto the chamber (13) and thus onto the pump space (34), the volume of the pump space (34) decreases because the chamber (13) is compressed. In this way, the pressure in the chamber increases and air is blown off into the atmosphere via the second one-way valve (16), as a result of which the gas volume and thus the pressure in the socket decreases.

In another embodiment of the invention, the abovementioned pump chamber (13) is integrated into an elastic stump cover or liner belonging to the socket. It is designated by (66) in the figures relating to the illustrative embodiments and is connected to the atmosphere via one-way valves in a manner similar to that described above.

In addition to the abovementioned advantages, the arrangement of the pump in the foam of a flexible pump chamber of variable volume in the interior (8) of the socket (2) also has the benefit that the restoring force, which in a preferred embodiment is provided by an insert (13) of elastic open-cell foam, does not have to work against the pressure difference inside the socket and the atmosphere, and instead it is necessary only to overcome the pressure difference resulting from losses of flow and throttling losses on the nonreturn valves and the flow channels. Therefore, with the teaching of the present invention, the elastically restoring element can be made considerably weaker and therefore also less expensive and of lighter weight.

By means of the underpressure that is generated, better adherence of the prosthesis is achieved and a suction action is exerted on the stump. The latter has the effect of counteracting the flow of interstitial fluid under the loading pressure during use of the prosthesis and of thus preventing a reduction in the stump volume or even of increasing the volume. As a result, the abovementioned gap between stump and prosthesis becomes smaller, and the pump action decreases and sometimes stops completely. As soon as there is no gap any longer present, however, there is also no longer any air between stump and socket, and a pump is superfluous. For correct functioning of a prosthesis equipped according to the invention, it goes without saying that the prosthesis socket must be impermeable to air and also that additional measures have to be taken, which measures are known per se and are not part of the subject matter of the present invention, in order to prevent entry of air through the proximal end of the prosthesis socket. In the case of below-knee prostheses, for example, this can be easily achieved by means of a hose-like elastic cover made of an airtight material being pulled with elastic pretensioning from the outer face of the prosthesis socket, over the knee and onto the skin of the thigh and bearing on the latter without a gap. Above-knee stumps, naturally with a large proportion of soft tissue, can be sealed off against entry of air by suitable design of the socket and without any additional measures, the suction action in this case depending to a great extent on the stability of the stump volume.

Preferred embodiments of the invention are explained in detail below on the basis of specific examples. It should be stressed that the embodiments shown are only preferred solutions for practical implementation of the invention and that the invention is not limited to the examples shown. Other arrangements and designs of individual components, and another combination thereof, may also meet the purpose of the invention.

The detailed description of the invention is made with reference to graphic depictions of the illustrative embodiments. Developments of the invention form the subject matter of the dependent claims. Combinations to which no illustrative embodiment is expressly directed must also be regarded as being claimed.

### DESCRIPTION OF THE FIGURES

FIG. 1 shows an amputation stump, in the area of the lower leg, in a corresponding below-knee prosthesis having the features of claim 1 of the invention, composed of a prosthesis socket with an artificial foot connected via an adapter piece and an extension element, the pump according to the invention with one-way valves, and an airtight cover from the outer face of the socket to the thigh. The socket and the hose-like cover are cut away in the sagittal plane in order to show the pump. The stump fits into the prosthesis socket free of clearance.

FIG. 2 shows the prosthesis from FIG. 1 in sagittal section, the stump being enveloped in a known cup-shaped elastic cover or liner.

FIG. 3 shows the prosthesis from FIG. 1 in sagittal section, which prosthesis is too wide for the stump and therefore no longer fits correctly, as often happens after the prosthesis has been worn for some time, as a result of displacement of body fluid caused by the loading pressure.

FIG. 4a shows the sagittal section through the wide prosthesis from FIG. 3, in the roll phase over the artificial front foot with loading shortly before the toes are lifted in the step cycle.

FIG. 4b shows the sagittal section through the wide prosthesis from FIG. 3, in the state of loading at the moment of heel impact.

FIG. 5 shows a preferred illustrative embodiment of the prosthesis socket with release pump. The pump chamber and the components interacting with it are shown in cross section. Of the prosthesis socket, a cross section is shown only through that part of the socket wall into which the pump chamber is built.

FIG. 6 shows a cross-sectional view of an embodiment of a one-way valve of the kind provided in the interior of the pump chamber from FIG. 5 at the intake opening.

FIG. 7 shows a cross-sectional view of an illustrative embodiment of a release valve for use in conjunction with a hose.

FIG. 8 shows the non-sectioned view of the side of a pump chamber according to the invention directed toward the socket wall.

FIG. 9 shows a plan view of the inner face of the socket wall in the area where the pump chamber is fitted.

FIG. 10 shows a longitudinal section through a below-knee prosthesis socket, in which the pump chamber as claimed in claim 24 is integrated in a liner, and the restoring force is provided by an open-cell foam inside the pump chamber.

FIG. 11 shows a longitudinal section through the prosthesis socket from FIG. 10, in which the outwardly facing wall of the pump chamber integrated in the liner is connected to the inner face of the socket wall by a Velcro connection.

### DETAILED DESCRIPTION OF THE INVENTION

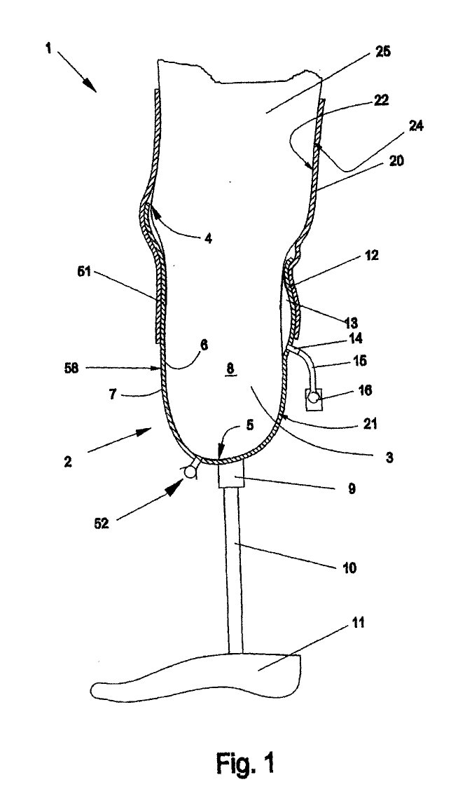

FIG. 1 illustrates the invention, using the example of a below-knee prosthesis (1) which is shown cut away in the sagittal plane. The socket (2), which receives the stump (3) and is adapted to it, is substantially cup-shaped with an open end (4) at the top, and with a closed, lower end (5) directed toward the ground. The socket (2) has an inwardly directed inner face (6) and an outwardly directed outer face (7). The inner face (6) forms a cup-shaped space (8) that receives the stump (1). The socket (2) is connected to an artificial foot (11) via an adapter piece (9) which is secured on the socket (2) and which has a tubular extension part (10). In the illustrative embodiment of the invention shown here, the rear (dorsal) socket wall has, in the upper (proximal) area, an elongate oval bulge (12) which forms a space for receiving a pump chamber (13). Although the bulge (12) is not absolutely necessary in all cases, it is advantageous for avoiding pressure sores. It is only as deep as is necessary to ensure that the flexible pump chamber (13), which will be described in more detail below, fills the space formed by the bulge (12) in the compressed state and in this way does not protrude into the interior (8) of the socket (2) and cause pressure sores on the stump (3). The pump chamber (13) is connected in an airtight manner to a tubular channel (14) which passes through the wall of the socket (2) and which in this way forms a flow channel from the interior of the pump chamber and through the socket wall. This flow channel opens into a further hose-like channel (15), which in turn establishes a flow connection to a one-way release valve (16), which will be described in more detail below. To avoid air from the upwardly open proximal end (4) getting into the gap between the skin of the stump (21) and the inner face (6) of the socket, an elastic cover (20) made of a moldable material, for example silicone, PUR or TPE, is provided in a known manner and bears, on the one hand, with its inner face (22) on the outer face (7) of the socket (2) and forms therewith a sealing surface, while, on the other hand, a second sealing surface is formed by means of the cover (20) bearing tightly on the skin (24) of the thigh (25) and molding itself thereto. As has already been mentioned, the depicted illustrative embodiment of the invention relates to a prosthesis for use after a below-knee amputation. However, the invention is not limited to a specific amputation height and instead can also be used analogously in prosthetic fixtures for other amputation heights.

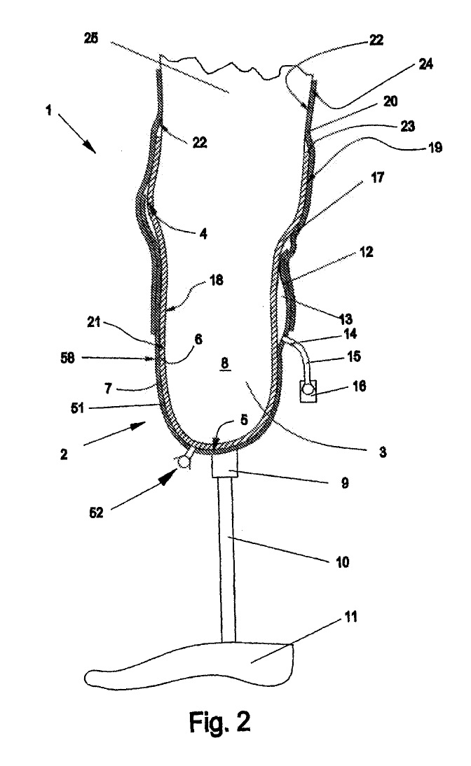

In terms of the invention, it is not important whether the stump is fitted into the socket with the skin bare or whether the stump carries a protective or cushioning cover (liner) (17) made of a moldable material, for example of an elastomer, advantageously of silicone, PUR or TPE, as is shown in FIG. 2, similarly in a section in the sagittal plane. Various designs of such liners are known to a person skilled in the art. Since these elastic liners mold tightly to the skin of the stump and there is therefore no air gap between the skin of the stump and the inner face (18) of the liner (17), the underpressure generated in the socket interior (8) by the pump (13) is transmitted to the stump even when the liner (17) is not gas-permeable. It goes without saying that, when using a liner, it is also necessary to ensure that no air from the environment can pass into the socket, in particular into the possible gap between the inner face (6) of the socket (2) and the outer face (19) of the liner (17), or between the inner face (18) of the liner (17) and the skin (21) of the stump. Therefore, the hose-shaped cover (20) expediently extends from the outer face (7) of the socket (2) beyond the proximal end (23) of the liner (17) as far as the skin (24) of the thigh (25). However, since the use of a known liner (17) is not essential to the invention, the stump together with the optionally used conventional liner is seen as a unit. Therefore, when the outer face of the stump or the skin of the stump is referred to, this can also signify the outer face of a conventional and known liner.

This does not apply to the embodiment of the invention which is discussed further below and in which the pump according to the invention is integrated into a liner, for which reason the liner is a necessary component part of the prosthesis and is therefore to be seen as one unit together with the socket.

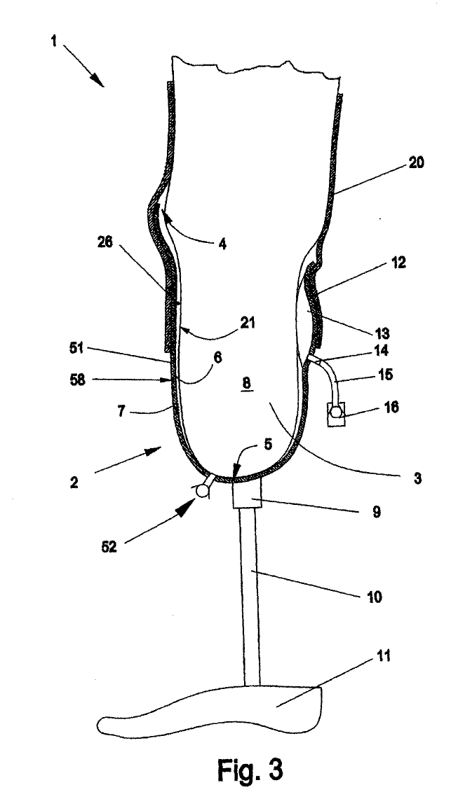

The volume enclosed by the interior (8) of the socket (2) is adapted individually to the volume of the stump of the prosthesis wearer at the time the prosthesis is fitted. When the stump (3) is inserted fully into the socket, the whole interior (8) should be filled. However, since it is a known fact that the stump volume is not stable over the course of time and in general decreases as a result of the pressure exerted on the skin during loading of the prosthesis, even when the prosthesis has been worn for only a short time, many people wearing leg prostheses experience the problem of the prosthesis socket quickly becoming too wide. FIG. 3 shows such a situation, on the basis of the prosthesis already shown in FIG. 1, in sagittal section. Between the skin (21) of the stump (3) and the inner face (6) of the socket (2), a gap (26) is shown that is filled with air. Even when the socket is not explicitly too wide and the skin bears fully on the inner face (6) of the socket (2) during static loading, the forces and moments that arise during the use of the prosthesis additionally have the effect that soft tissue parts are displaced. Since this displacement also takes place upwardly in the proximal direction, there are always times, while walking or running with a conventional prosthesis, when the skin (21) of the stump loses contact with the inner wall (6) of the socket, thus leading to reduced skin pressure and even to a gap (26).

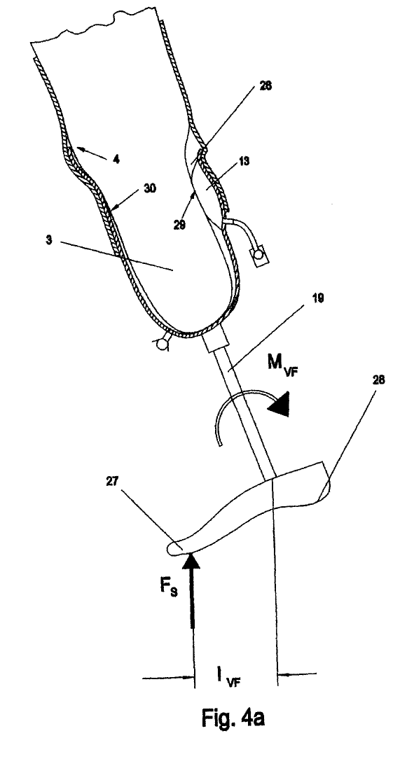

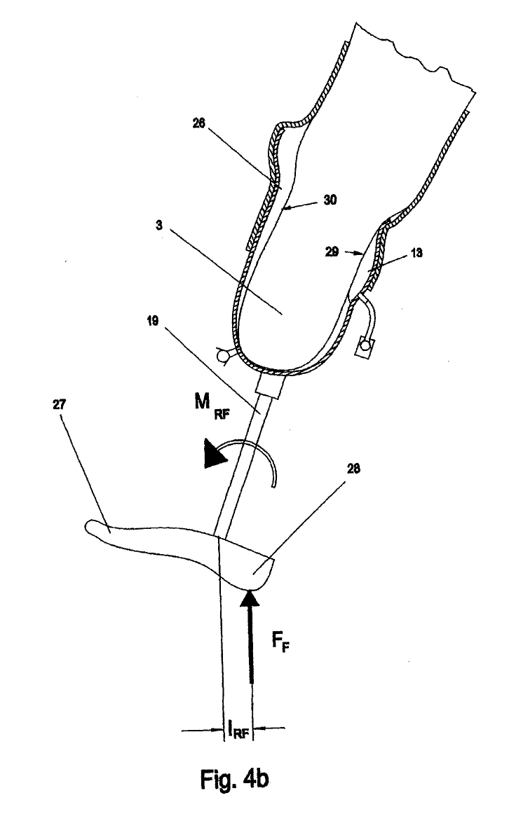

To illustrate this, FIG. 4a and FIG. 4b show the two load situations that arise during a step cycle shortly before lifting the front (27) of the foot (FIG. 4a) and shortly after placing the heel (28) on the ground. When pushing off from the front of the foot during a step cycle, the static and dynamic loading force Fₛ at the front of the foot and the front foot lever l_{VF} from the loading point to the center line of the extension tube (19) cause a reaction moment M_{VF} which acts as a bending moment on the stump/prosthesis system and which leads to the proximal prosthesis edge (4) being pressed heavily on the shin bone (30) in the area of the knee, while the pressure on the calf decreases. In FIG. 4a, this is indicated by a gap (26) shown in the dorsal area of the socket, that is to say between the calf (29) and the rear inner wall (6) of the socket. The pump chamber (13) is not compressed and, because of the elastic insert, is inflated to a maximum possible volume.

Conversely, an opposite bending moment M_{RF} arises at the time of heel impact, as a result of the loading force F_{F} and the rear foot lever l_{RF}, and forces the proximal socket area away from the area of the shin bone (30), as is indicated in FIG. 4b. The calf (29) is in this way pressed more firmly against the pump chamber (13), which is consequently compressed and thus decreases in volume.

With his knowledge of these loading profiles and his understanding of the function of the pump chamber (13) according to the invention in a prosthesis socket (2), a person skilled in the art will soon see that the favorable position of the pump chamber according to the invention may differ on an individual basis and is dependent on the nature of the stump, on the soft-tissue coverage of the bone structures, on muscle function, and other aspects. A position is preferred at which the relative movements arising during use between stump and socket, also referred to by a person skilled in the art as stump/socket pseudarthrosis, are at their greatest, such that the path of actuation of the pump, as will be explained in more detail below, is as great as possible. In the case of a below-knee prosthesis, this is the proximal socket area, as can be clearly seen in the embodiment of the invention from FIGS. 4a and 4b.

In the illustrative embodiments shown, a pump chamber (13) is provided in the dorsal and proximal area of the socket, for example. It is of course also possible to accommodate one or more pump chambers at other locations in the socket. For example, it has also proven particularly advantageous to place a respective elongate pump chamber of ca. 5 cm to 12 cm in length and ca. 4 cm to 8 cm in width, in the socket on both sides of the shin bone. If several pump chambers are provided for the invention, they can all operate independently of one another or can also interact in a cascade form. The latter arrangement is not illustrated and is only described here. It is useful when, for reasons relating to their structure, the pump chambers have a certain dead space, i.e. a volume that cannot be further reduced. According to the laws of gas physics, the dead space limits the maximum possible pressure difference between the intake side of the pump and the release side which, in the pumps associated with the present invention, is the side with the higher pressure, because the air volume in the pump chamber at the initial pressure fills the dead space at a pressure which is at most equal to the pressure on the outside, in the present case the atmospheric pressure. In this state therefore, when the pump is actuated, the air in the pump is just cyclically compressed and expanded again. By prefilling an at least second pump chamber with the air volume of at least one upstream pump chamber, the possible pressure difference of the system is thus increased in the presence of a dead space and at otherwise constant conditions. In the latter case, at least 2 pump chambers (13) are arranged one after another in series, in such a way that the one-way valve (16) at the outlet of the first pump chamber (13), which is arranged for example in the area of the calf (29), serves at the same time as an inlet valve (37) at the inlet opening (36) of an at least second pump chamber (13) which, for example, is arranged opposite in the shin bone area (30). Only the outlet opening (38) of this second pump chamber is then connected in the described manner to the atmosphere via a flow channel (14) through the socket wall (51) and outward via a hose (15) and an outflow valve (16). This arrangement has the advantage that the amount of air in the second pump chamber is increased by the displaced air from the first pump chamber. In this way, the possible pressure difference from the pump action is greater with a fixed dead space.

In an extended foam of the invention, which is not illustrated specifically here, provision is made to increase the volume of the underpressure space. This is of advantage when small leakages are to be compensated for over quite a long period of time and is achieved by the fact that a further airtight space in the form of a hollow container made of substantially stiff material is connected to the interior of the socket (8) via a flow channel which is of sufficiently small diameter to ensure that there is no danger of the skin of the stump being sucked into the channel. The additional volume thereby created acts as it were as a buffer and delays the pressure increase upon delivery of air into the socket through leakages or the like.

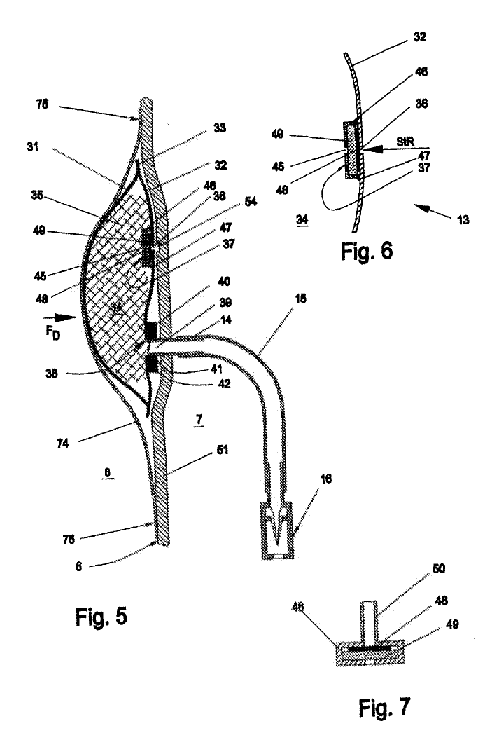

The function of an individual pump chamber in connection with the invention will be described in detail with reference to FIGS. 5, 6 and 7.

It has already been mentioned several times that an aim of the invention is to ensure that air present in a gap between the outer face of an amputation stump and the inner face of a prosthesis socket, and if appropriate in a buffer volume in communication with the latter, can be transported out of the socket, and thereby to generate an underpressure in said gap. To displace a volume of air counter to a higher pressure, mechanical work has to be physically expended, which requires energy. It will be clear from what has been said above that this energy is made available through relative movements between socket and stump resulting from the static and dynamic forces and moments that arise during use of the prosthesis. As is known, the product of force and travel equals energy. Therefore, in order to implement the invention, a system has to be provided which, by means of a force exerted over a defined actuation path, is able to compact gas molecules, i.e. to increase the pressure, in order to move the molecules counter to the atmospheric pressure from a lower pressure level into the atmosphere.

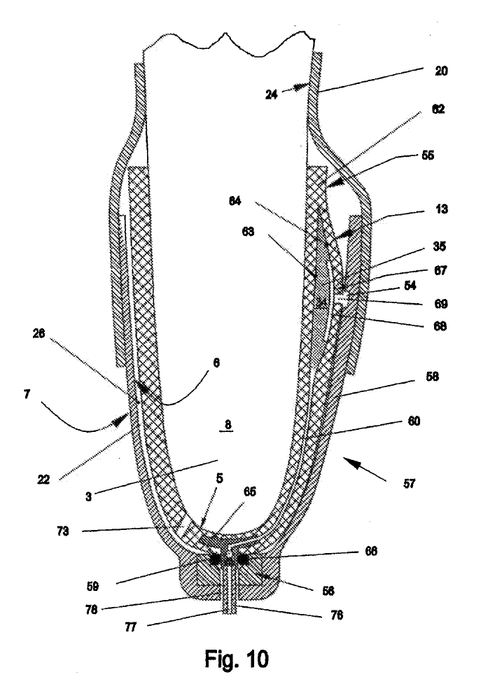

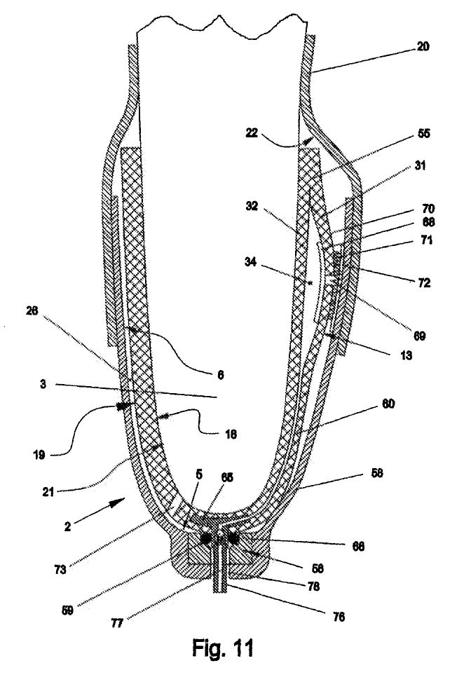

In a preferred embodiment of the invention, this is achieved by using a pump chamber (13) that encloses a pump space (34). The pump chamber (13) is made from a flexible, air-impermeable membrane, and possible embodiments and preferred arrangements are described in more detail with reference to FIGS. 5, 6 and 7 and also to FIG. 10 and FIG. 11. Although the inventive effect of the prosthesis socket with active air release is in principle the same regardless of whether the at least one pump, as described in FIGS. 1 to 9, is built in a fixed position into a stiff socket, or whether the pump is part of a removable elastic part of an otherwise substantially stiff socket according to

FIG. 10 and FIG. 11, the two embodiments are described separately below.

In the illustrative embodiments shown in FIGS. 1 to 4, the pump chamber (13) has an elongate oval shape, of which the longer side extends approximately parallel to the vertical axis of the prosthesis. The short side is ca. 5 to 8 cm and the longer side ca. 8 to 12 cm.

FIG. 5 shows an enlarged cross section of the pump from the preceding figures. In the simplest form, the pump is composed of an inwardly directed layer (31) and of an outwardly directed layer (32) of flexible film, which films are welded or cohesively connected in an airtight manner about the entire circumferential edge, as is indicated by (33) in FIG. 5. This welding means that the chamber thus formed is able to inflate as shown. Examples of suitable film materials would be, among others, PUR or PVC which, as is known, can be easily connected by HF or thermal welding methods. Since the pump chamber is intended to take in air and release it again, the two welded films (31 and 32) have to enclose a space (34) whose capacity is greater than zero and is here designated as the pump space. To do so, it is therefore necessary for the two films to be mounted at a certain distance from each other. This is achieved most simply by the pump space (34) being filled at least partially with a foam material (35). Since, according to the invention, the pump is intended to be actuated repeatedly in cycles, and since the above-described stump/socket pseudarthrosis in this illustrative embodiment can only exert a pressure force FD in the arrow direction and not in the opposite direction, a resilient element must be provided in order to return the pump chamber to its starting shape. This purpose is again served by the foam (35), which is expediently an open-cell foam in order to ensure that air can flow through. At a suitable place on the pump chamber, an opening (36) is provided which, in the example shown, is simply a hole in the film through which air can flow from the interior of the socket (8) via a one-way valve (37), which will be described in more detail below, into the interior (34) of the pump chamber (13). In the example shown, the opening (36) points toward the inner face (6) of the socket wall (51). As will be explained in more detail below, a groove (54) is provided as flow channel, such that the opening cannot accidentally become blocked. At another location there is a second opening (38), through which air can pass from the interior (34) into a flow channel (39) when the volume of this space is reduced on account of a force FD acting on the flexible film chamber. In the example shown, this channel (39) is composed of a tube (14) which is connected in an airtight manner to the socket wall (51), for example by lamination, such that no air can get in between the outer face of the tube and the socket wall. The tube (14) protrudes by ca. 5 mm into the interior (8) of the socket (2). A ring (40) made of an elastic material, e.g. rubber, is provided in an airtight manner on the pump chamber (13), for example by adhesive bonding to the film (32). The internal diameter (42) of the ring (40) is flush with the opening (38) in the pump chamber and is slightly smaller than the external diameter of the tubular flow channel (14). With the elastic ring (40), the pump chamber (13) can simply be fitted onto the inwardly protruding part of the tube (41) and fixed with a friction fit and form fit and in an airtight manner, if the surface pairing is suitable and if the elastic pretensioning on account of the differences in diameter is great enough. Alternatively, it is of course also possible for the ring (40) to be connected in an airtight manner to the tube end (41) with a cohesive fit, for example by adhesive bonding. The flow channel (39) is continued on the outer face (7) of the socket (2) by a flexible hose (15), which establishes a connection to a further one-way valve (16). On the inner face (6) of the socket (2) in this example, a protective cover (74) is provided which is made, for example, of an air-permeable textile spanning the entire surface of the pump chamber (13), and which, for example, is connected to the socket wall in the area (75) with a cohesive fit or form fit, for example by adhesive bonding. The purpose of the cover is to protect the pump chamber from mechanical damage, and it acts at the same time as a dust filter and thus prevents impaired functioning of the one-way valves (37) and (16) caused by dirt. For the sake of simplicity and to make the drawings clearer, the protective cover is not shown in the other figures.

FIG. 6 shows an illustrative embodiment of a one-way valve (37), of the kind arranged in the interior (34) of the pump chamber (13) in the area of the inlet opening (36) in such a way that a flow connection is established from the socket interior (8) through the opening (36) in the film of the pump chamber, through the one-way valve (37) and through at least one further opening (45) on the one-way valve. The direction of flow is indicated by an arrow (StR) in FIG. 6. The housing (46) of the one-way valve (37) is connected to the inner face of the film (32), for example by adhesive bonding or welding in the edge area (47). A membrane (48), for example of silicone film, covers the opening (36) and is pressed gently onto the opening (36) by an open-cell, flexible foam insert (49). In this way, a one-way valve known per se is formed. As soon as the pressure in the interior (34) of the pump chamber is higher than in front of the inlet opening (36), the membrane (48) is pressed harder against the opening (36), such that no air can pass through. In the reverse case, that is to say when the pressure in the interior (34) of the pump chamber (13) is lower than in front of the inlet opening (36), the pressure difference lifts the membrane (48) from the opening (36), and a flow takes place. For the best possible functioning of the invention, the one-way valve (37) has to be configured in such a way that a very low pressure difference Δp is sufficient for opening in the direction of flow (StR). This can be achieved by measures that are known per se in one-way valves. The configuration shown in FIG. 6 is just one illustrative embodiment of a one-way valve and does not in any way limit the present invention to this configuration.

FIG. 7 basically shows the one-way valve from FIG. 6 with a tube attachment (50) for use as outflow valve (16) in conjunction with the flow channel (15).

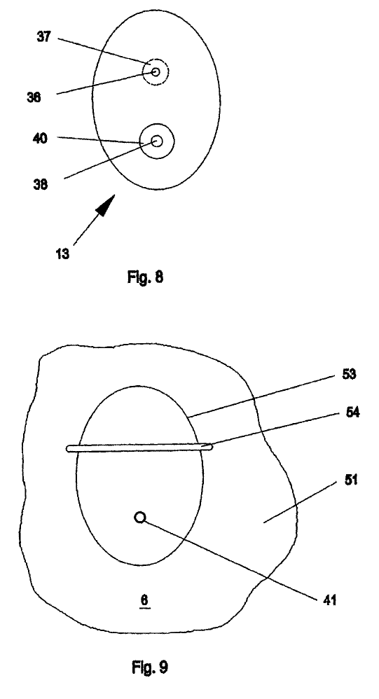

FIG. 8 shows the side of the prosthesis socket (2) directed toward the inner face (6) in an illustrative embodiment of the pump chamber (13) according to the invention with the above-described sealing ring (40) around the opening (38), and the opening (36) with the one-way valve (37) which is directed toward the inner face and which, as an element that cannot be seen in this view, is indicated by a broken line. In this illustrative embodiment, as has also already been shown in FIG. 5, the opening (36) does not point directly to the inner face of the socket (8) but to the inner face (6) of the socket wall (51). This has proven advantageous, since it avoids a situation where the opening is accidentally closed by the skin of the stump (21) or by its cover (17).

FIG. 9 shows, in a front view, a detail of the inner face (6) of the socket wall (51) in the area of the position of the pump chamber (13), which is indicated by the contour line (53). A recessed groove (54) is present in the socket wall. The groove (54) is located in the area where the suction opening (36) of the pump chamber (13) comes to lie and it protrudes past the area of extent (53) of the pump chamber (13). It is thus ensured that the suction opening (36) of the pump chamber cannot be covered and sealed off by the otherwise smooth inner wall (6) of the socket (2). It goes without saying that, in addition to the groove shown in the example, other measures are also possible to avoid accidental blocking of the flow opening (36), for example insertion of a coarse textile between the pump (13) and the inner wall (6) of the socket.

FIG. 10 shows another embodiment of the invention. It only depicts a cross section through the socket, with the stump located therein. The other component parts of a prosthesis have not been shown, since these are not essential to an understanding of the invention. A pump liner made of an elastic and flexible material, for example of silicone, TPE or PUR, and a connecting means (56) and an airtight cup-shaped container (58) which is made of a substantially stiff material and, as in the embodiment from FIGS. 1 to 5, is adapted to the type of amputation and to the conditions of the stump in accordance with the known rules of prosthesis design, together form, as one unit, a prosthesis socket (57) according to the invention. The releasable connection between the stiff socket part and the flexibly elastic portion is necessary for fitting the prosthesis in place. Since the high static friction between flexibly elastic materials and the human skin is one of the reasons that makes it impossible to simply slide into a flexible sleeve of this kind, the flexible pump liner (55) has to be rolled onto the stump in a known manner like a conventional liner before fitting the stump into the stiff socket part. The connecting means (56) serves to connect the flow channel (60), (77) of the pump liner to the atmosphere, but at the same time to prevent entry of air into the interior (8) of the cup-shaped container (58). In the view according to FIG. 10, the size of the pump liner (55) is chosen such that it is always slightly smaller in circumference than the stump (3), such that it has to be rolled with elastic pretensioning onto the stump (3) and bears fully on the surface of the stump (3). After application of the pump liner (55), the stump is inserted into the stiff socket part (58). At the distal end of the pump liner there is a tubular pin (76), which forms a flow channel (77) that is connected to a further flow channel (60) via a one-way valve (59), which in the drawing is outlined as a spring-loaded ball in a valve seat and, being a known element, is not described in any more detail. At its end remote from the one-way valve (59), the for example tubular flow channel (60) opens into the pump space (34) of a pump chamber (13) of variable volume. The pump chamber (13) and the flow channel (60) are located within the wall (62) of the pump liner (55). For this purpose, the wall (62) is divided in the area of the pump space (34), such that approximately half of the wall thickness forms the inner face (63) and outer face (64), respectively, of the pump space (34). In a similar way, the flow channel (60) is formed, for example by insertion, during vulcanization of the pump liner, of a spacer that does not adhere to the basic material. The spacer can be removed after the vulcanization, resulting in a tubular channel. Insertion of a hose would be another possible way of forming the flow channel (60) in the wall of the pump liner. The channel is to be hose-shaped and should at all times remain open during normal use of the prosthesis. A lumen of 1 to 3 mm in diameter is sufficient. It is also possible to produce the liner from several layers, in which case the layers are cohesively connected at those places where no cavities or flow channels are wanted. Another production variant would be for the pump chamber and the necessary flow channels to be prefabricated and to be inserted into a tool for production of the pump liner and in this way to be vulcanized in or embedded in plastic. To secure the tube (76) in the pump liner, a rigid part (65) is cast in at the distal end, which rigid part (65) allows passage of the flow channel (60), receives the one-way valve (59) and has the tube (76) secured in it.

The connecting means (56) is arranged as centrally as possible at the closed end (5) of the cup-shaped container (58) and is composed of the tubular flow channel (76) and of the seal (66), which is here designed as an O-ring, and creates an airtight connection between the rigid adapter piece (65) at the distal end of the pump liner (55) and the socket base (5). With this arrangement, the opening (78) in the socket base is sealed off against entry of air as soon as the stump (3) with the pump liner (55) is located completely in the container (58).

The air gap (26), which is exaggerated in the drawing for the sake of clarity, is thus sealed off from the outer face of the prosthesis, and no air can penetrate into it. As has been described previously, the proximal end of the prosthesis socket is provided again with a flexible hose-shaped cover (20) for sealing the outer face (7) of the socket (58) with the skin of the thigh (24). In the pump space (34) there is an elastically resilient element, for example an open-cell foam (35), which elastically restores the pump chamber in the manner described above. Accordingly, the function of this illustrative embodiment of the invention is analogous to the illustrative embodiment shown above. As a result of the stump/socket pseudarthrosis and the presence of an appreciable gap (26), the volume of the elastically resilient pump chamber is changed during use of the prosthesis, by means of being compressed by the stump and then, as the external mechanical pressure abates, being enlarged again by the elastic insert (35). The air located in the pump space is pressed out through the flow channel (60) and the one-way valve (59) into the channel (77) and thus to the outside of the prosthesis socket. A further one-way valve (67), which in the simplest case is formed by a membrane (68) in front of an opening (69) that again connects the pump space (34) to the interior of the socket (8), allows the pump space (34) to be refilled with air from the socket interior (8) when the elastic insert (35) presses the elastic and flexible walls of the pump space apart again and the volume of the pump space thus increases. As has already been described in FIG. 5, it may be necessary to ensure that the opening (69) is not covered by the socket wall, for example by providing a recess (54) in the surface of the inner face (6) of the socket wall (51). By cyclic repetition, the air pressure in the interior (8) of the socket (57) is repeatedly lowered. The explanations given above apply analogously and in full to this embodiment of the invention. Thus, in this embodiment too, several pump chambers with corresponding valves and channels can be provided, working individually or in cascade. As has been described above, an additional buffer volume can also be provided.

FIG. 11 shows an embodiment of the invention similar to that in FIG. 10. In contrast to the above example, the pump space (34) is not provided with an elastically resilient element for restoring the pump, and instead the outer face (70) of the outwardly directed wall (31) of the pump chamber in the pump liner (55) is equipped with a self-adhesive means (71, 72), as a result of which a releasable connection to the inner face (6) of the dimensionally stable socket part (58) can be established. This can be achieved in the simplest way by means of a Velcro connection, in which the outer face (70) of the pump liner (55) in the area of the pump is provided for example with a layer of loops (71), and corresponding hook elements (72) are placed on the inner face (6) of the hard socket part (58) in this area. To fit the prosthesis in place, the pump liner (55) is once again first pulled or rolled onto the stump (3), and a separating film (not shown) is placed between the Velcro elements, with the aid of which the stump can be easily slid into the rigid socket part. After the separating film has been pulled out in the proximal direction, the Velcro connection according to the invention is obtained as described above. By virtue of the fact that the outwardly facing wall (31) of the pump space (34) now bears on the inner face (6) of the rigid socket wall, and that the inner wall (32) of the pump chamber (13) remains bearing tightly on the stump as a result of the elastic pretensioning and as a result of underpressure effects, the volume of the pump chamber (13) changes, and therefore the volume of the pump space (34) changes, upon relative movements between the stump and the socket in the direction perpendicular to the main plane of the pump chamber (13), thereby generating the pump action. Since the tensile forces arising in the Velcro connection only have to overcome the elastic tensioning of the outer wall (31) of the pump chamber, the required adhesive force of the Velcro connection is so low that it is easy to tear open the connection for the purpose of removing the prosthesis.

In other possible embodiments, not illustrated here, the pump liner in the area of the pump chamber is made of a flexible material with restoring force, or a shaped part made of corresponding flexible material with restoring force is located in the pump space (34) of the pump chamber (13). In such embodiments in which the pump chamber, after deformation by external forces, returns to its original state simply as a result of the restoring force of the material after the deforming forces have been removed, it is possible to dispense with a foam insert or a connecting means for transmitting tensile forces, as described in FIG. 11. Instead of an open-cell foam insert, and in analogy with the above configurations, the elastic restoring of the pump space (34) of the pump chamber (13) of the pump liner (55) can be achieved by inclusion of an air chamber of smaller volume.

In FIG. 10 and FIG. 11, a flow channel (73) is additionally shown which allows pressure compensation between the space limited by the inner face (18) of the liner and the skin (21) of the stump and the space between the outer face (19) of the liner, the inner face (6) of the rigid socket part (58) and the inner face (22) of the cover (20).

1. A cup-shaped prosthesis socket (2) composed at least of a
substantially dimensionally stable container (58) made of an airtight material, with an open end (4) at the top and a closed end (5) at the bottom, for receiving an amputation stump (3), and with a means for avoiding entry of air into the gap (26) between the skin (21) of the stump (3) and the inner face (6) of the socket at the proximal edge area of the socket,
characterized in that
at least one air pump is arranged in the interior (8) of the prosthesis socket (2) in direct proximity to the stump (3), which air pump
is composed of at least one chamber (13) whose volume can be changed by external forces,
has an inlet valve (37) in the socket interior,
is equipped with at least one flow channel (14, 60, 77), which establishes a flow connection from the pump space (34) to the outer face (7) of the socket (2), and with a corresponding outlet valve (16, 59)
and which is actuated by deformation caused by forces from relative movements between stump and socket during use of the prosthesis, such that air is pumped out from the interior (8) of the socket (2) into the atmosphere.

2. The prosthesis socket as claimed in claim 1, characterized in that the at least one air pump has, by comparison to its extent tangential to the socket wall (51), a small thickness in the direction perpendicular to the socket wall.

3. The prosthesis socket as claimed in claim 1, characterized in that the pump chamber (13) is accommodated in a recess (12) in the socket wall (51).

4. The prosthesis socket as claimed in claim 1, characterized in that the pump chamber (13) enclosing the pump space (34) is formed by flexible film (31, 32) welded along its edge.

5. The prosthesis socket as claimed in claim 1, characterized in that the pump chamber (13) enclosing the pump space (34) is formed from a closed envelope of elastic material.

6. The prosthesis socket as claimed in claim 4, characterized in that an air-permeable elastic insert (35) is provided inside the pump space (34) in order to increase the volume of the pump chamber (13) after a reduction in volume by external forces.

7. The prosthesis socket as claimed in claim 1, characterized in that the pump chamber (13) enclosing the pump space (34) is formed as an airtight cavity made from an at least partially deformable flexible material with shape memory and with suitable restoring force.

8. The prosthesis socket as claimed in claim 1, characterized in that the pump chamber (13) enclosing the pump space (34) is made from an elastic or flexible material, and means (71, 72) are provided by which not only volume-reducing pressure forces but also volume-increasing tensile forces can be transmitted to the walls (31, 32) or (63, 64) of the chamber.

9. The prosthesis socket as claimed in claim 1, characterized in that the two valves (16, 37) are one-way valves that both permit only one direction of flow from the interior (8) of the socket (2) out into the atmosphere.

10. The prosthesis socket as claimed in claim 1, characterized in
that the air pressure in a gap between the outer face (21) of the stump (3) and the inner face (6) of the socket (2) is lowered to below atmospheric pressure by the pump.

11. The prosthesis socket as claimed in claim 1, characterized in that the one-way valve (37), which allows air into the pump space (34), is provided in the interior of the pump space (34) and is cohesively connected to one wall (32, 64) of the pump space in the area of the flow opening (36, 54).

12. The prosthesis socket as claimed in claim 11, characterized in that the one-way valve is a flutter valve made of film.

13. The prosthesis socket as claimed in claim 12, characterized in that the membrane (48, 68) of the flutter valve (37, 67) is kept closed with slight pretensioning by an elastic, open-cell foam (49).

14. The prosthesis socket as claimed in claim 13, characterized in that the flutter valve (37, 67) is located in its own housing (46) and in this way is not mechanically influenced by the foam (35) in the pump space (34).

15. The prosthesis socket as claimed in claim 1, characterized in that the pump is mounted with a sealing ring (40), which completely surrounds an opening (38) and which is connected in an airtight manner to the wall (32) of the pump chamber (13), onto a part (41) of a tubular flow channel (14) which protrudes into the socket interior and which extends through the socket wall (51) and is connected to the latter in an airtight manner.

16. The prosthesis socket as claimed in claim 15, characterized in that the flow channel (14) through the socket wall is a tube and can be continued externally by a hose (15).

17. The prosthesis socket as claimed in claim 1, characterized in that the outer one-way valve (16) is likewise a flutter valve.

18. The prosthesis socket as claimed in claim 1, characterized in that the outer one-way valve (16) is what is called a duckbill valve.

19. The prosthesis socket as claimed in claim 6, characterized in that the elastic insert in the pump space (34) is formed by a prefilled air chamber.

20. The prosthesis socket as claimed in claim 19, characterized in that the pressure in the air chamber changes and can therefore be adjusted individually.

21. A prosthesis arrangement with a prosthesis socket (2) as claimed in claim 1, characterized in that the wall (32) of the pump space directed toward the inner face (6) of the socket wall (51) is connected securely to the inner face (6) of the socket wall (51), and the opposite inwardly facing wall (31) is connected releasably to a band that encircles the stump at least at the height of the pump chamber.

22. The prosthesis arrangement as claimed in claim 21, characterized in that the encircling band is formed by that part of an elastic liner (17) which, during use of the prosthesis, is directly adjacent at least partially to the pump.

23. The prosthesis arrangement as claimed in claim 21, characterized in that the releasable connection of the liner (17) to the inwardly facing wall (31) of the pump chamber (13) is a Velcro connection.

24. The prosthesis socket as claimed in claim 1, characterized in that the chamber (13) of changeable volume is integrated into the wall (62) of a flexible stump cover (55).

25. The prosthesis socket as claimed in claim 24, characterized in that the flexible cover (55) is made from an elastomer, such as silicone, TPE or PUR.

26. The prosthesis socket as claimed in claim 24, characterized in that a flow channel (60) from the pump chamber (13) to a connecting means (56) is integrated into the wall (62) of the flexible cover (55).

27. The prosthesis socket as claimed in claim 26, characterized in that the connecting means (56) establishes a releasable but airtight connection between the flow channel (60) and the atmosphere.

28. The prosthesis socket as claimed in claim 27, characterized in that the connecting means (56) is arranged distally in the socket base.

29. The prosthesis socket as claimed in claim 24, characterized in that the outlet valve (59) is built into the connecting means (56).

30. The prosthesis socket as claimed in claim 1, characterized in that an additional buffer volume is connected in terms of flow to the interior (8) of the socket.
